(19) United States
(12) Patent Application Publication
(10) Pub. No.: US 2004/0143269 A1
(43) Pub. Date: Jul. 22, 2004

(54) Cutting device for cutting bone tissue

(75) Inventors: Frank Pude, Rodenberg (DE); Hartmut Louis, Garbsen (DE); Ludger Kirsch, Seelze (DE); Carl Joachim Wirth, Isernhagen KB (DE); Staphan Schmolke, Hannover (DE)

Correspondence Address: CROWELL & MORING LLP, INTELLECTUAL PROPERTY GROUP, P.O. BOX 14300, WASHINGTON, DC, 20044-4300, US

(73) Assignees: Universitaet Hannover, Hannover (DE); Medizinische Hochschule Hannover, Hannover (DE)

(21) Appl. No.: 10/683,669

(22) Filed: Oct. 17, 2003

Related U.S. Application Data
(63) Continuation of application No. PCT/DE02/01226, filed on Apr. 4, 2002.

(30) Foreign Application Priority Data
Apr. 19, 2001 (DE) 101 19 328.9

(51) Int. Cl. A61B 17/16

(52) U.S. Cl. 606/79

(57) ABSTRACT

A cutting device (1) for cutting bone tissue (3), which is particularly intended for correcting malpositions, and which enables a segment of the bone tissue (3) to be cut along a predetermined cut surface (5). The cutting device (1) produces a high pressure fluid jet (2) which is guided by a control program stored in a control unit (4) to produce a cut surface (5) having a desired configuration, for example, a circular arc shape. The resulting bone parts (6, 7), created in this manner, can be fixed in a corrective position (8) with little effort and without significant loss of material. A collector (9), which is equipped with a supply line (14) for supplying a rinsing liquid and with a suction line (13), is provided for collecting the fluid from the fluid jet (2). Collector (9) simultaneously serves to reduce the energy remaining in the high-pressure fluid jet (2). A cut surface (5) having a freely selectable shape can be produced by the cutting device (1) and can be obtained without any difficulty by using automated handling systems. The cutting device of the invention enables a significant broadening of the field of use and of the design freedom for an orthopedic surgeon together with a use that preserves bone tissue.

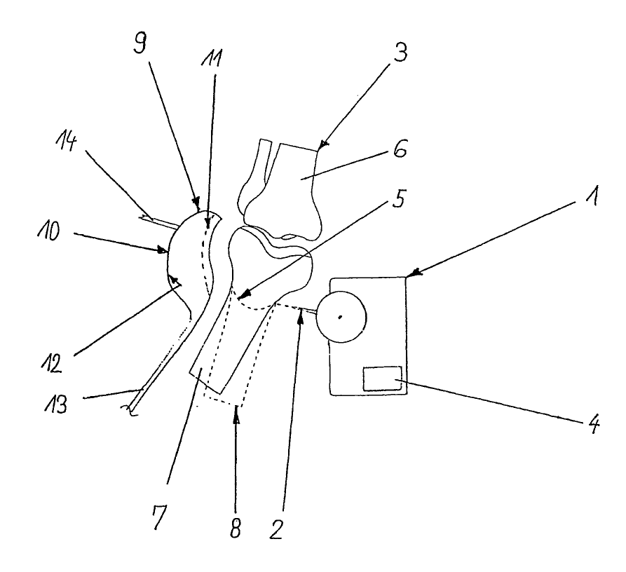

### CROSS REFERENCE TO RELATED APPLICATIONS

This application is a continuation of International Patent Application No. PCT/DE02/01226, filed Apr. 4, 2002, designating the United States of America, and published in German as WO 02/085223, the entire disclosure of which is incorporated herein by reference. Priority is claimed based on Federal Republic of Germany Patent Application No. DE 101 19 328.9, filed Apr. 19, 2001.

### BACKGROUND OF THE INVENTION

The invention relates to a cutting device for bone tissue, particularly for correcting malpositions, which can be used to cut a segment of the bone tissue along a predetermined cut surface.

The osteotomy procedures used today in surgical orthopedics have changed little with respect to the cutting devices which are used, notwithstanding the changed requirements in the last few decades. Whereas in the first half of this century the chisel and the manually guided saw, for example Gigli's saw, were used, the compressed-air-driven oscillating saw with fixed saw blade is employed almost exclusively today. Other cutting devices, such as high-speed trephines, are used only for special indications, e.g., in spinal decompression procedures. However, this device is not used for bone cutting in the narrower sense but rather for local bone ablation.

Cutting the bone tissue in the living body is at the core of many orthopedic procedures and is therefore extremely important. Particularly the implantation of endoprostheses requires great precision in preparing the seat for the implant. The primary stability of cement-free prostheses depends directly on the fit between the prosthesis and the osseous seat and is the basis for a permanently stable implant. On the other hand, any damage to the edge zones of the bone tissue will result in delayed osseous integration and a shortened life of the implant.

In component implantation of knee endoprostheses, a precise anatomical alignment is of paramount importance for the physiological functioning and thus the life of the joint replacement. When the conventional method is used, the intraoperative alignment of the saw templates for the implant seat is done manually. During the procedure, the surgeon can orient himself/herself only by the local anatomical conditions. The intramedullary target systems which are used partly show deviations that are too large for precise placement; the cuts are performed manually with the oscillating saw. The saw templates further offer only a small support surface for the saw blade. Due to the potential for errors in each of these surgical steps, malalignments of the implant components do occur and are associated with a significantly reduced load capacity and shortened life of the implant.

A further limitation of the oscillating saw as well as the chisel is due to the cut geometries that are necessarily produced, which are limited to creating two-dimensional, i.e., flat surfaces. As a result, the shaping of the prosthesis as practiced today is adapted to these limitations and is therefore significantly restricted compared to other desirable shapes which would in all likelihood create substantially better conditions, especially with respect to the permanent load capacity.

Hence, the quality of the component implant is substantially limited by the limited usability of the available tools and aids and, in addition, substantially depends on the skill of the surgeon. When bones are mechanically cut, particularly using the oscillating saw, one of the drawbacks observed is the heat created by friction. If the temperature of 57° C., which is critical for proteins, is exceeded, irreversible necrosis occurs along the cut surfaces. Such edge necrosis has a negative effect on osteointegration.

A further drawback in mechanical processing is that it largely excludes the use of automated handling systems in surgery because an essential prerequisite, the avoidance of large process forces and high pressures against the bone, cannot be reliably achieved. Nor is the laser widely used for cutting bone tissue in surgical orthopedic practice. Many studies involving C0₂ lasers showed partly extensive carbonization zones along the cut edges and thermally damaged transition zones. In living bone, these zones act like foreign bodies, impede callus formation and thus delay healing. Experiments with other laser systems having different wavelength ranges, e.g., the YAG laser and the excimer laser, showed clearly reduced thermal damage zones. However, due to the lower power output, the working speed was far below that of conventional osteotomy techniques. Ablation of biological hard tissue using these methods cannot be done with the clinically necessary speed. It is therefore limited to special applications, e.g., in dental and maxillary surgery, to prepare bone canals for anchoring dental implants.

Furthermore, “Waterjetting technology in orthopedic surgery: cutting experiments on bone and bone cement,” C. Brandt, E. Hille, M. Honel, H. Louis, R. Rentzsch, 1998, describes a method for removing an implant or a prosthesis from the bone tissue. Here, a substantial portion of the bone cement can be removed using a high-pressure liquid jet. As a result, the tissue is protected because little force is required to remove the implant. A comparatively thin liquid jet is optimal for this purpose because it achieves a large depth of cut with little loss of material. Depending on the different material properties of the bone tissue on the one hand and the bone cement on the other, the parameters of the liquid jet are adjusted such that damage to the bone tissue is in any case excluded. Operating errors that could injure the patient are thus excluded, even in manual operation.

It is further known in the art to select the parameters of the high-pressure liquid jet such that the liquid jet selectively cuts only parenchymal organs (liver, kidney) while the more solid vessels are preserved and can be safely ligated by the surgeon with little loss of blood. Another application area, although in a clearly smaller scope, is neurosurgery. Due to the difference in characteristics, however, this technique cannot be transferred to the cutting of bone tissue because damage to adjacent tissues and organs cannot be excluded in view of the parameter adjustments that are required to cut through bone tissue.

### SUMMARY OF THE INVENTION

It is an object of the invention to provide a cutting device that substantially improves the process of cutting bone tissue in orthopedic surgery.

Another object of the invention is to provide a cutting device for bone tissue which satisfies increased requirements regarding the quality and accuracy of the cut surface.

A further object is to provide a cutting device for bone tissue which allows flexibility in use in view of individual requirements.

These and other objects are achieved in accordance with the present invention by providing a cutting device for cutting a segment of bone tissue along a predetermined cut surface, wherein said cutting device produces a high-pressure fluid jet, which can be guided along a freely selectable cut surface of the bone tissue and to which pharmacologically compatible abrasive agents can be added to enhance the cutting performance.

Further refinements and preferred aspects of the invention are set forth hereinafter.

Thus, according to the invention, a cutting device is provided, which produces a high-pressure fluid jet that can be guided along a freely selectable cut surface of the bone tissue. Pharmacologically compatible abrasive agents may be added to enhance the cutting performance. This makes it possible for the first time to adapt the contour of the cut surface to the individual requirements in order to produce an optimal fit, e.g., for prostheses. At the same time, it is possible to produce a cut surface enabling a correction of malpositions through joining in a different position or orientation without loss of material. The application of tensile stress or compressive stress for correction can be eliminated. No significant force is required to guide the high-pressure fluid jet either manually or by an automated handling system because no pressure against the bone is required. The easy control of the cut surface further makes it possible to orient the jet in such a way that undesirable damage to adjacent tissue can be avoided. This is accomplished, for example, in that the high-pressure fluid jet, after passing through the bone tissue, escapes into the environment where its energy is reduced and the fluid, which is essentially water, is collected.

A particularly advantageous embodiment of the cutting device is achieved if the cutting device is configured to cut the bone tissue along a cut surface having a freely selectable shape. This makes it possible to substantially reduce the loss of material when bone tissue is cut, particularly in preparation of fitting a prosthesis. In particular, the surgeon is not limited to individual flat surfaces that are aligned at a fixed angle in relation to one another. In practice, therefore, a broader scope of application and flexibility in design is made possible to meet the respective requirements. The cut surface can also have a contour that is optimized for the healing process and for permanent connection with the prosthesis.

Another particularly advantageous embodiment of the invention is achieved if the cutting device is equipped with a control unit used to determine the desired cut surface. As a result, it is no longer left to the skill of the surgeon to achieve an optimal cut surface. Instead, the cut surface is determined in advance with the aid of the control unit. This increases not only the precision but also the speed of the operation. The control programs can be created either based on existing models or individually. Even deviations that occur during the cutting process can be immediately converted into a correction value to ensure an optimal result.

It is advantageous if the control unit has a control program for cutting a surface in the shape of a circular arc. This makes it possible to fix the bone parts separated along the cut surface in a new position different from the original position without a clearance or gap appearing between the surfaces of the bone parts. The cut surface can moreover be dimensioned such that the bone parts can be fixed relative to one another in only a single changed position. Thus, the cut surface already defines the new relative position or orientation of the bone parts, so that potential errors in joining the bone parts are avoided. At the same time, the healing process is clearly accelerated.

Yet another particularly advantageous embodiment of the invention is achieved if the cutting device has means for reducing the energy of the high-pressure fluid jet on a side of the bone tissue to be cut that faces away from the high-pressure fluid jet. This prevents damage to tissue or organs and, in addition, prevents the uncontrolled discharge of the fluid into the environment. The means can be configured in such a way that the tissue parts or organs involved are protected from the high-pressure fluid jet by being shielded or displaced from the immediate zone of influence of the high-pressure fluid jet.

It is especially advantageous if the means comprises a deflector with a suction line for the fluid. This makes it possible reliably to remove the fluid even under unfavorable ambient conditions by discharging it through the suction line. This prevents interference with or even a reaction effect on the cut surface and avoids any need for an undesirably large collecting device.

In an especially suitable embodiment of the invention, the means comprises a supply line for a rinsing liquid to ensure that particles and tissue parts are reliably removed.

### BRIEF DESCRIPTION OF THE DRAWING

The invention will be described in further detail hereinafter with reference to an illustrative preferred embodiment shown in the accompanying drawing FIGURE, which is a schematic representation of a cutting device for cutting bone tissue according to the invention.

### DETAILED DESCRIPTION OF PREFERRED EMBODIMENTS

The invention is susceptible to various embodiments. To further illustrate the basic principle of the invention, one such embodiment is depicted in the accompanying drawing, which schematically depicts a cutting device 1 according to the invention. The cutting device 1 produces a high-pressure fluid jet 2, particularly a water jet to which an abrasive agent is added to enhance the cutting performance. It is used to cut human bone tissue 3 along a cut surface 5 (indicated by a broken line) that is predetermined with the aid of a control unit 4. In the example shown, the cut surface 5 follows a circular arc to facilitate correction of a malposition along the cut surface 5 by fixing the bone parts 6, 7 in a different corrected position 8 indicated by a broken line without a substantial loss of material. The cutting device 1 is further equipped with means 9 for reducing the energy of the high-pressure fluid jet 2 on a side of the bone tissue 3 to be cut that faces away from the high pressure fluid jet 2. For this purpose, the means 9 comprises a deflector 12 that is provided with an indentation 10 and covered with a screen 11. Deflector 12 is connected to a suction line 13 for removing the fluid. In addition, a supply line 14 for a rinsing liquid is provided, so that particles can be reliably removed.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A cutting device for bone tissue for cutting a segment of bone tissue along a predetermined cut surface, wherein said cutting device produces a high-pressure fluid jet, which can be guided along a freely selectable cut surface of the bone tissue and to which pharmacologically compatible abrasive agents can be added to enhance the cutting performance.

2. A cutting device according to claim 1, wherein the cutting device is configured for cutting bone tissue along a cut surface having a freely selectable shape.

3. A cutting device according to claim 1, wherein the cutting device is provided with a control unit for determining the desired cut surface.

4. A cutting device according to claim 3, wherein the control unit has a control program for cutting a cut surface in the shape of a circular arc.

5. A cutting device according to claim 1, wherein said cutting device includes means for reducing the energy of the high pressure fluid jet on a side of the bone tissue to be cut that faces away from the high-pressure fluid jet.

6. A cutting device according to claim 5, wherein said energy reducing means comprises a deflector equipped with a suction line for evacuating the cutting fluid.

7. A cutting device according to claim 5, wherein said energy reducing means is provided with a supply line for supplying a rinsing liquid to a cut area.
